(19) United States
(12) Patent Application Publication
(10) Pub. No.: US 2015/0313611 A1
(43) Pub. Date: Nov. 5, 2015

(54) UNDERCUTTING BONE DRILLS AND METHODS OF USE

(71) Applicant: Howmedica Osteonics Corp., Mahwah, NJ (US)

(72) Inventors: Felix Omar Torres O'Farrill, Gurabo, PR (US); Charles McCartney, Denver, CO (US); Kyle Craig Pilgeram, San Jose, CA (US); Ross Callison, Denver, CO (US); José Raúl Marchand, San Juan, PR (US); Anthony P. Napolitano, Chappaqua, NY (US)

(73) Assignee: HOWMEDICA OSTEONICS CORP., Mahwah, NJ (US)

(21) Appl. No.: 14/268,685

(22) Filed: May 2, 2014

(51) Int. Cl. A61B 17/16

(52) U.S. Cl. CPC A61B 17/1617

(57) ABSTRACT

A bone cutting device for forming an undercut in bone. The device includes a rotatable body having an inner surface and outer surface. The inner surface defines an aperture. The inner and outer surfaces are disposed opposite each other and define a sidewall. The sidewall includes a recess extending from the outer surface into the aperture. A cutting arm is cantilevered to the sidewall and disposed within the recess. The cutting arm includes a flexible depression extending into the aperture. An actuating member is longitudinally slidable within the aperture such that sliding the actuating member longitudinally interfaces with the flexible depression and pushes the cutting arm radially outwardly.

### BACKGROUND OF THE INVENTION

Soft tissue structures, such a ligaments and tendons, connect multiple anatomic components together. Whether the connection is bone-to-bone, muscle-to-bone, or some other linkage, these soft tissue structures are often, if not permanently, subject to tension forces. Injuries can partially or completely sever such structures leading to immobility and/or dysfunction of the anatomic components. In one example, a shoulder injury may tear a portion of the rotator cuff from its connection to bone, leading to instability of the shoulder joint and causing the naturally tensioned tendon to slacken.

In some instances surgery may be needed to repair the soft tissue structure, which often involves pulling the soft tissue back into its natural state of tension and into a position for healing, which may take weeks or months. Maintaining the soft tissue in a healing position and in a constant and consistent state of tension may be beneficial in allowing the soft tissue to heal as closely to its natural state as possible and to prevent any healing progress from becoming undone.

Generally, an anchoring support and, optionally, a filament attached to the anchoring support, is utilized in soft tissue reparation. The soft tissue may be tensioned by the filament or otherwise anchored into position by the anchoring support, which can be a bone anchor suitable for being positioned within a prepared bore hole in bone. However, obtaining and maintaining the desired soft tissue tension and position for the entire healing period may be difficult. Constant forces applied to the filament and anchoring support throughout the healing period may cause the anchoring support to unseat or shift, causing potential excess wear to the filament and/or slackening of the filament, potentially reducing the efficacy of the repair or completely undoing the repair.

### BRIEF SUMMARY OF THE INVENTION

The following disclosure generally relates to an undercutting drill for forming an undercut hole in bone to receive a filamentary suture anchor. In one aspect of the present disclosure, a bone cutting device for forming an undercut includes a rotatable body having an inner surface and an outer surface. The inner surface defines an aperture. The inner and outer surfaces are disposed opposite each other and define a sidewall. The sidewall has a recess extending from the outer surface into the aperture. A cutting arm is cantilevered to the sidewall and disposed within the recess. The cutting arm includes a flexible depression extending into the aperture. An actuating member is longitudinally slidable within the aperture such that sliding the actuating member longitudinally interfaces with the flexible depression pushing the cutting arm radially outwardly.

Additionally, the depression may include a concave portion having a first radius of curvature and a convex portion having a second radius of curvature. Applying a force to the concave portion may reduce the size of the first and second radius and increases the length of the cutting arm. The rotatable body may include a cutting tip disposed at a first end of the rotatable body and may be configured to remove bone when a first force is applied to a second end of the rotatable body. The bone cutting device may also include a spring coupled to the actuating member at the second end of the rotatable body. The spring may bias the actuating member in a first direction and may be compressible in a second direction by a second force.

Continuing with this aspect, the cutting arm may be formed from at least three cuts that extend through the sidewall of the rotatable body. The at least three cuts may include a first cut, a second cut, and a third cut. The first cut may begin at a first point and extend along the rotatable body in a first direction. The second cut may begin at the end of the first cut and extend along the rotatable body in a second direction. The third cut may begin at the end of the second cut and extend along the rotatable body in a third direction and end at a second point. The rotatable body may have a longitudinal axis and a transverse axis orthogonal to the longitudinal axis. The first direction may be about 5 degrees or less with respect to the longitudinal axis of the rotatable body, the second direction may be about 5 to 10 degrees with respect to the transverse axis, and the third direction may be about 0 degrees with respect to the longitudinal axis. 8. The first cut and third cut may be disposed on the same side of second cut. The first cut and third cut may also be disposed distally of the second cut. Further, the first point may be offset from the second point in a proximal-distal direction.

In another aspect of the present disclosure, a bone cutting device for forming an undercut in bone includes an elongate shaft having a longitudinal axis, a plurality of cutting arms cantilevered to the elongate shaft and having a first position and second position, and a bushing slidable over the elongate shaft and at least a portion of the cutting arms. The bushing is adapted to slide between a distal position over the cutting arms, which moves the cutting arms into the first position and a proximal position, and away from the cutting arms, which moves the cutting arms into the second position.

Additionally, the cutting arms may extend away from the longitudinal axis in the second position and converge toward the longitudinal axis in the first position. Further, the cutting arms may be biased towards the second position. In some embodiments, at a first temperature, the cutting arms may be biased towards the first position, and at a second temperature, the cutting arms may be biased towards the second position.

Continuing with this aspect, the bushing may be biased towards the distal position over the cutting arms. The cutting arms, in the first position, may form a distal cutting tip, which may include a distal-most point and a cutting surface along each cutting arm. The cutting tip may also include an enlarging taper expanding to a location on the cutting tip proximal of the distal-most point and have a width larger than a width of the elongate shaft. The bushing may be prevented from sliding distally to a position beyond the width of the cutting tip that is larger than the width of the elongate shaft. When the cutting arms are in the second position, the cutting surface may be substantially parallel with the longitudinal axis of the elongate shaft.

In a further aspect of the present disclosure, a bone cutting device for forming an undercut includes a tubular drill body having an inner surface and outer surface defining a sidewall therebetween. The bone cutting device also includes a drill tip disposed at one end of the tubular drill body and a cutting arm formed from the sidewall by at least three cuts through the sidewall. The cutting arm includes a cantilevered end, a free end, and a cutting surface disposed between the cantilevered and free ends. Additionally, the bone cutting device may include a plunger having a length. The plunger may be disposed within the tubular drill body and may be coupled to the tubular drill body at a location along the length of the plunger.

In yet a further aspect of the present disclosure, a method for forming an undercut in bone includes driving a drill tip of a bone cutting device into the bone a predetermined depth by a first push force applied to an end of the cutting device. The method also includes deploying a cutting arm radially from the bone cutting device upon the application of a second force larger than the first force to the end of the cutting device.

Additionally, the cutting device may include a tubular drill body and a plunger disposed within a channel of the tubular drill body. Further, the first and second push forces may be applied to the plunger. The plunger and tubular drill body may be coupled by a spring. The first force may not be sufficient to compress the spring and the second force may be sufficient to compress the spring. A cutting arm, which may be cantilevered to the tubular drill body, may be coupled to the plunger, and the first force may not be sufficient to buckle the cutting arm and the second force may be sufficient to buckle the cutting arm.

In still another aspect of the present disclosure, a bone cutting device for forming an undercut includes a drill tip having a longitudinal axis passing therethrough, a shaft having an offset portion, and a first and second bend. The offset portion is offset from the longitudinal axis by the first and second bend. Additionally, the first and second bend may each include a straight portion having a length of about 1 to 3 mm.

### BRIEF DESCRIPTION OF THE DRAWINGS

The features, aspects, and advantages of the present invention will become better understood with regard to the following description, appended claims, and accompanying drawings in which:

FIG. 1A is a front cutaway view of a first embodiment of an undercutting drill.

FIG. 1B is a partial cross-sectional view of the undercutting drill taken along line A-A of FIG. 1A.

FIG. 1C is a cross-sectional view of an undercut hole formed by the undercutting drill of FIG. 1A.

FIG. 2A is a front view of another embodiment of an undercutting drill in an undeployed configuration.

FIG. 2B is a front view of the undercutting drill of FIG. 2A in a deployed configuration.

FIG. 3A is a front view of yet another embodiment of an undercutting drill in a first configuration in bone and having a cutting tip.

FIG. 3B is a close-up view of the cutting tip of FIG. 3A in the first configuration.

FIG. 3C is a front view of the undercutting drill of FIG. 3A in a second configuration in bone.

FIG. 3D is a close-up view of the cutting tip of FIG. 3B in the second configuration.

FIG. 4 is a perspective view of a further embodiment of an undercutting drill.

FIG. 5A is a front cutaway view of a still further embodiment of an undercutting drill.

FIG. 5B is another front cutaway view of the undercutting drill of FIG. 5A.

FIG. 5C is a side cross-sectional view of the undercutting drill of FIG. 5A taken along the midline of the undercutting drill.

FIG. 6A is a front cutaway view of another embodiment of an undercutting drill.

FIG. 6B is a side cross-section view of the undercutting drill of FIG. 6A taken along the midline of the undercutting drill.

### DETAILED DESCRIPTION

As used herein, the term “operator” refers to any individual operating the instruments and/or performing the methods disclosed herein, which can include a clinician, nurse, assistant, doctor, a robot assisted by a person, or the like who may utilize the present invention. The terms “proximal” and “distal” are used herein relative to the operator, such that “proximal” means closer to the operator and “distal” means further from the operator. The term “soft tissue” refers to any tissue such as ligaments, tendons, capsule, cartilage, meniscus, skin, muscle, adipose tissue and the like. The term “bone” refers to bone or hard tissue, and includes cortical and cancellous bone. Also, as used herein, the terms “about,” “generally” and “substantially” are intended to mean that slight deviations from absolute are included within the scope of the term so modified.

The devices, instruments, kits, and methods of the present disclosure are contemplated for use in both arthroscopic and open surgical procedures.

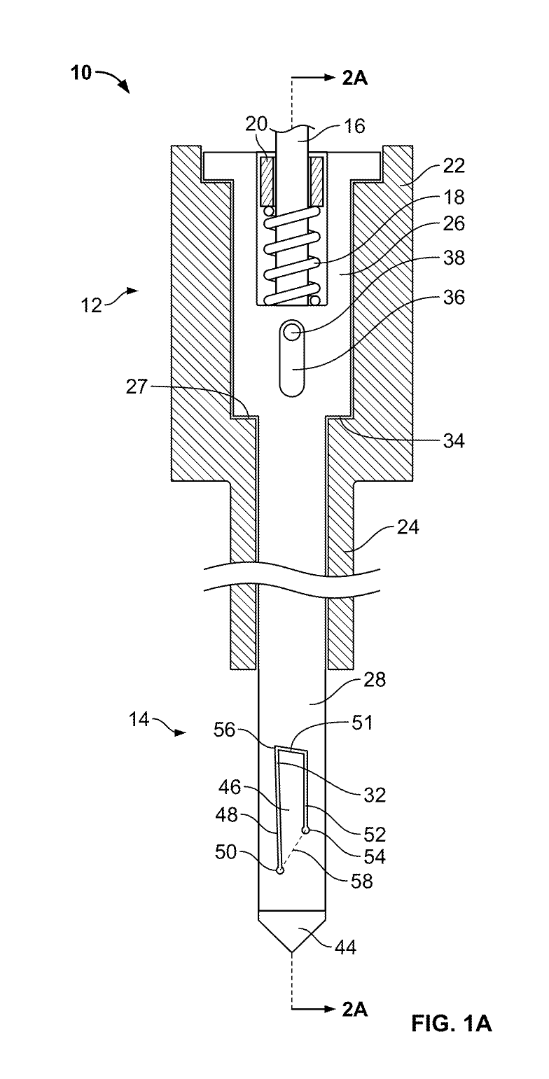

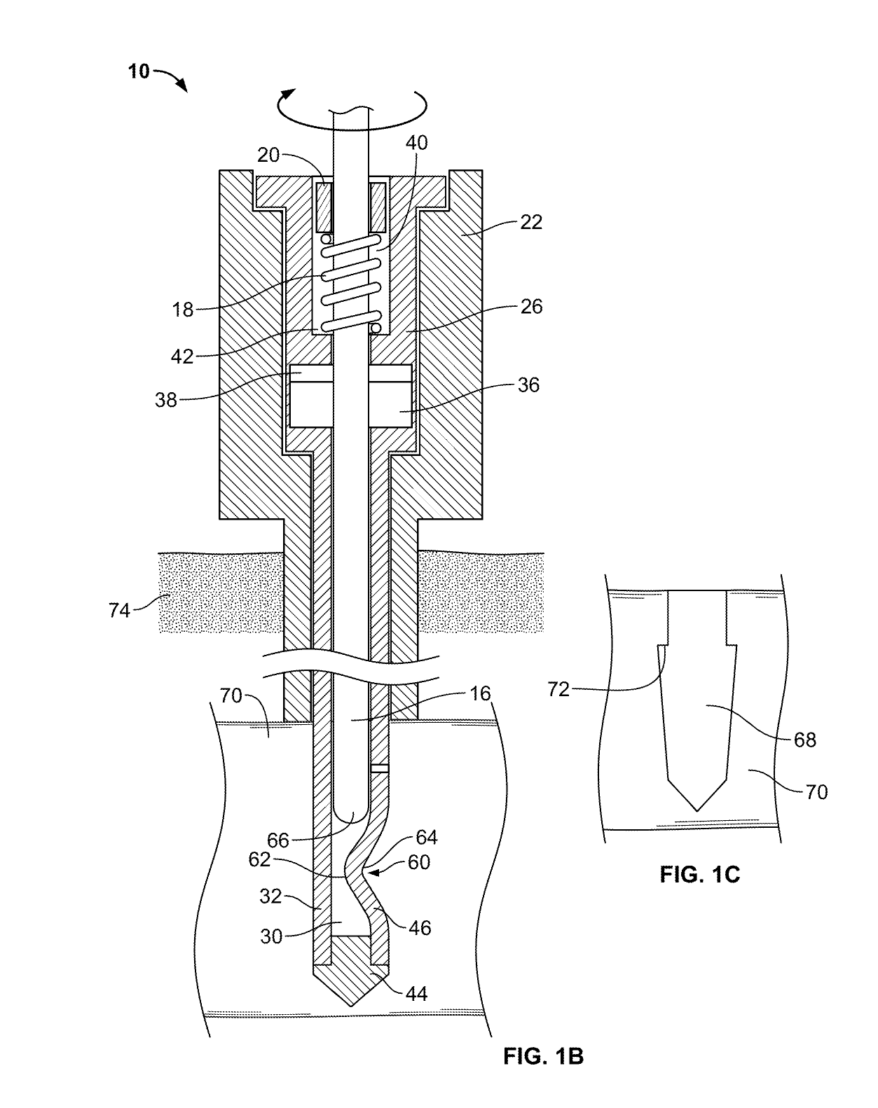

FIGS. 1A and 1B depict a first embodiment undercutting drill 10, which generally includes a drill guide 12, a drill body 14, an actuating member 16 or plunger 16, a spring 18, and a retainer 20. The drill guide 12 is annular and includes a first portion 22, a second portion 24 and a substantially cylindrical passageway extending therethrough to define an inner diameter of the first and second portion 22, 24. The inner diameter of the first portion 22 is concentrically arranged with and larger than the inner diameter of the second portion 24 to form a shelf 27. The first portion 22 and second portion 24 each include an outer diameter, wherein the outer diameter of the first portion 22 is larger than the outer diameter of the second portion 24. In some embodiments, the second portion 24 is long relative to the first portion 22 and can act as a cannula for arthroscopic surgery or alternatively fit within a cannula used in arthroscopic surgery. The first portion 22 and second portion 24 can be mechanically joined, such as by interference fit, or formed together to create a monolithic structure, such as by machining the drill guide out of a slug of raw material.

The drill body 14 includes a proximal portion 26 and a distal portion 28. The proximal portion 26 and distal portion 28 are tubular and both include an aperture 30 that extends through the proximal portion 26 and into the distal portion 28, thereby defining a sidewall 32 between the aperture 30 and an outer surface of the drill body 14. The proximal portion 26 has a larger outer diameter than the distal portion 28 to form a shoulder 34 that is capable of rotatably engaging the shelf 27. The outer diameter of the proximal portion 26 is sized to fit within the first portion 22 of the drill guide 12 but not the second portion 24 of the drill guide 12. The outer diameter of the distal portion 28 is sized to fit within the first portion 22 and second portion 24 of the drill guide 12. Thus, when the drill body 14 is inserted into the passageway of the drill guide 12, the shoulder 34 can abut the shelf 27 to prevent distal movement of the drill body 14 with respect to the drill guide 12 while allowing rotational movement of the drill body 14 with respect to the drill guide 12.

The proximal portion 26 includes a pair of slots 36 that extend from the aperture 30 into the sidewall 32. The slots 36 are elongated in a proximal-distal direction along the proximal portion 26 to form an ovular shape in order to receive a substantially cylindrical pin 38 that is sufficiently large to transfer a torque to the drill body 14, from a source, such as an electric drill (not shown), and through plunger 16, sufficient to penetrate cortical bone. The length of each slot 36 is generally determined by the desired travel distance of the plunger 16 within the aperture 30 of the drill body 14, which is discussed further below. The slots 36 are disposed directly opposite each other such that a single pin may extend into both slots 36.

The aperture 30 extending through the proximal portion 26 expands radially outwardly near the proximal end of the proximal portion 26 to create a clearance space 40 for the spring 18, or other biasing mechanism, and retainer 20. The formation of the clearance space 40 may also create a retaining surface 42 for retaining and opposing distal movement of the spring 18 and for transferring a push force from the plunger 16 and spring 18 to the drill body 14, as discussed further below.

The distal portion 28 is generally long relative to the proximal portion 26 and includes a drill tip 44 and a cutting arm 46. The drill tip 44 can be any drill tip configured to penetrate cortical and cancellous bone and is disposed at the distal end of the distal portion 28. The cutting arm 46 is created from and cantilevered to the sidewall 32 generally by cutting, such as by laser cutting, through the sidewall 32 of the drill body 14 and into the aperture 30.

The cutting arm 46 may be formed from the sidewall 32 in any shape desired. For example, as illustrated in FIGS. 1A and 1B, the formation of the cutting arm 46 is achieved by three cuts. The first cut 48 begins at a first circular hole 50, which is circular to reduce stress concentrations, and extends along the drill body 14 in a proximal direction at an angle of about 1 to 5 degrees with respect to a longitudinal axis of the distal portion 28. The second cut 51 begins at the end of the first cut 48 and extends along the drill body 14 in a transverse direction at about a 5 to 10 degree angle with respect to a transverse axis that is orthogonal to the longitudinal axis of the distal portion 28. The third cut 52 extends from the end of the second cut 51 in a distal direction at about a 0 degree angle with respect to the longitudinal axis of the distal portion and terminates at a second circular hole 54. While cutting arm 46 as illustrated is preferred, it is envisioned that the various shapes, lengths, angles, number of cuts, and like designations may be altered as desired.

Cutting surfaces (not shown) are located along the first cut 48 and second cut 51, and a cutting tip 56 is located on the cutting arm 46 at the junction between the first and second cut 48, 51. The cutting surfaces may be dull or blunt or may be sharpened to form an edge once the cutting arm 46 is created. In some embodiments, when the first and second cuts 48, 51 are performed to form cutting arm 46, such cuts may be made perpendicular to the surface of the distal portion 28 to create a flat edge along first and second cuts 48, 51. In other embodiments, first and second cuts 48, 51 can be performed at an oblique angle with respect to the surface of the distal portion 28 so as to create an inclined cutting edge along first and second cuts 48, 51.

Continuing with this exemplary cutting arm 46, the first circular hole 50 and second circular hole 54 are offset from each other in a proximal-distal direction where the first circular hole 50 is disposed at a more distal location than the second circular hole 54. A pivot axis 58 connects holes 50 and 54 and defines a hinge about which the cutting arm rotates 46. The offset configuration of the first and second circular holes 50, 54 facilitates a twisting action of cutting arm 46 about the cutting arm's own longitudinal axis as the cutting arm 46 rotates about the pivot axis 58. This twisting action may facilitate exposure of the cutting surfaces and cutting tip 56 so that when the cutting arm 46 is extended from the drill body, the cutting tip 56 and cutting surface lead the remainder of the cutting arm and are placed at a sufficient cutting angle with respect to bone. Further, the twisting action may decrease the angle between the second cut 51 and transverse axis and help prevent the cutting arm 46 from bending along its length as it cuts through bone by providing multidirectional structural resistance.

An additional advantage of the offset configuration is resilience against bending and shearing forces at the pivot axis 58. As the cutting arm 46 cuts through bone, shearing and bending forces can create stresses at the pivot axis 58. As compared to a cutting arm with a first and second circular hole that are not offset in a proximal-distal direction, the offset configuration may reduce the stresses felt by the cutting arm 46 by having a pivot axis 58 with increased length and a pivot axis 58 angled with respect to the direction of the magnitude of the applied forces. Further, this angling of the pivot axis 58 shortens the moment arm helping to reduce bending stress.

As illustrated in FIG. 1B, this cutting arm 46 also can include a depression 60 that extends into the aperture 30. The depression 60 is a segment of the cutting arm 46 that is depressed to form a rounded convexity 62 that extends into the aperture and a rounded concavity 64 that is disposed opposite the convexity 64. The convexity 64 has a first radius and the concavity 62 has a second radius. The first radius is larger than the second radius, the difference of which may vary with the thickness of the cutting arm 60. Additionally, the radius of the convexity 62 may be varied to alter the rate at which the cutting arm 46 extends from the drill body 14 when engaged with the plunger 16.

The cutting arm 46, as well as the drill body 14, is constructed from a biocompatible material that is flexible yet resilient. In one example, the material may be a Nitinol alloy that increases in stiffness as temperature increases, such as by frictionally generated heat due to drilling. Other examples include titanium, titanium alloys, stainless steel, cobalt-chromium, tantalum, and niobium.

The flexibility of the material allows the cutting arm 46 to rotate about the pivot axis 58 and allows the concavity 62 to compress when engaged with the plunger 16 such that the first radius and second radius reduce in size, which translates into an increase in the cutting arm length 46. The resiliency allows the cutting arm 46 to sufficiently remove bone. Additionally, the roundness allows the cutting arm 46 to gradually swing outward from the drill body 14 as it is contacted by the plunger and provides resistance to bending along the length of the arm 46 as the cutting arm 46 is deployed.

The plunger 16 is an elongate shaft having a first end configured to connect to a torque applying device, such as an electric drill, and a second end 66 that may be rounded and blunt. The plunger 16 is sized to fit and move longitudinally within the aperture 30 of the drill body 14 and is long enough to extend beyond the depression 60 of the cutting arm 46. The plunger 16 includes a pin hole such that pin 38 can extend through the plunger in a transverse direction relative to a longitudinal axis of the plunger 16. The pin 38 can fit through the slots 36 of the drill body 14 and transfer torque from the plunger 16 to the drill body 14.

The spring 18 may be a helical spring capable of fitting over the first end of the plunger 16. In one example, the spring 18 may have a stiffness large enough to prevent the compression of the spring 18 when a push force sufficient enough to advance the drill body 14 through bone is applied to the plunger 16, yet small enough so that the spring 18 can be compressed by a larger push force applied by the operator. In other words, a push force delivered to the plunger 16 sufficient to allow the drill tip 44 to cut through bone to the desired depth can be smaller than the minimum force required to compress the spring 18. In another example, the spring stiffness may be such that the spring would not compress as the drill tip advances through bone in response to a push force, but would compress when the shoulder 34 abuts the shelf 27 and the entire push force is realized by an equal and opposite resistance force.

When the undercutting drill 10 is fully assembled, the drill body 14 resides within the substantially cylindrical passageway of the drill guide 12 such that the distal portion 28 of the drill body 14 is disposed in the second portion 24 of the drill guide 12 and the proximal portion 26 of the drill body 14 is disposed within the first portion 22 of the drill guide 12. The drill body 14 may be slidable and rotatable within the substantially cylindrical passageway. Sliding movement distally may be opposed by the abutment of the shelf 27 and the shoulder 34. When drilling into bone, the drill depth is generally determined by the difference of the distance between the shoulder 34 and the end of the drill tip 44 and the distance between the shelf 27 and the end of the second portion 24.

The plunger 16 resides and is slidable within the aperture 30 of the drill body 14. The pin 38 extends through the plunger 16 and into the slots 36 effectively preventing rotational movement of the plunger 16 with respect to the drill body 14 but allowing slidable, longitudinal movement of the plunger 16 with respect to the drill body 14. The length of the slots 36, in a proximal-distal direction in conjunction with the diameter of the pin 38, determines the length of travel of the plunger 16 within the drill body 14. As the plunger 16 is rotated by a torque applying device, the pin 16 is capable of transferring the torque to the drill body 14 which is rotatable within the drill guide 22.

The spring 18 is disposed within the clearance space 40 of the body 14 and surrounds a portion of the plunger 16. One end of the spring 18 is attached to and/or abuts the retaining surface 42 of the drill body 14 and the other end is attached to and/or abuts the retainer 20. The retainer 20 may be an annular collar that is connected to the proximal end of the plunger 16 proximal of the spring 18 for retaining the spring 18. A push force applied to the plunger 16 can be transferred from the retainer 20 to the spring 18 and then to the drill body 14 by the spring 18. A push force may be transferred to the drill body 14 without compressing the spring 18. However, where the drill body 14 is prevented from moving in a distal direction by the interference between the shoulder 34 and shelf 34, the spring 18 can be compressed between the retaining surface 42 and retainer 20.

In an alternative embodiment of undercutting drill 10, the cutting arm 46 can be a separate structure cantilevered to the sidewall 32 by mechanical means, such as by welding, and may be constructed from a material more flexible than the remainder of the drill body 14. In other embodiments, the cutting arm 46 can be formed by the three cuts as previously described but may differ in that the first and second circular holes 50, 54 may not be offset. In such a configuration, the first and second circular holes 50, 54 would both reside in a transverse plane that is orthogonal to the distal portion's longitudinal axis. In another embodiment, the cutting surfaces of the first and second cuts 48, 51 may be further refined to provide a serrated edge or a heat treated edge for cutting the bone. In yet another alternative, the cutting arm can be constructed using less than three cuts or more than three cuts, which would achieve variously shaped and/or oriented cutting arms as desired.

In some embodiments, the undercutting drill 10 may not have a plunger 16. In such an embodiment, the cutting arm 46 can be constructed from a memory metal material, such as Nitinol, that would be biased in a direction toward the aperture of the drill body 14 at room and body temperature, but would expand outward as the drill body 14 temperature is raised beyond body temperature.

In other embodiments, the depression 60 can have a convex portion 62 extending into the aperture 30 but not a concave portion 64 disposed opposite the convexity 62. Alternatively, the depression 60 can be separate structure such as a stud mechanically connected to the inside of the cutting arm 46, such as by welding or riveting.

In one alternative multiple undercutting drills 10 or multiple drill bodies 14 may be provided in a kit. In these kits, the drill bodies 14 may vary to allow the operator to adjust the size and depth of the undercut hole. For example, the distal portion 28 and cutting arm 46 may vary in size and length to provide the operator the flexibility to determine drill depth and undercut size. Additionally, such kits can be provided with variously sized bone anchors, such as for example filamentary anchors, such as Iconix® all suture anchor system (Howmedica Osteonics, Mahwah, N.J.), that each correspond to corresponding undercutting drills or drill bodies, such that where a particular size filamentary anchor is selected, a precisely sized undercutting drill may be selected along with that anchor. Other examples of filamentary anchors that may be implanted into an undercut hole formed by the instruments disclosed herein and that may be provided in a kit as just described are disclosed in U.S. application Ser. No. 13/783,804, filed Mar. 4, 2013; Ser. No. 13/303,849, filed Nov. 23, 2011; Ser. No. 13/588,586, filed Aug. 17, 2012; Ser. No. 13/588,592, filed Aug. 17, 2012; Ser. No. 13/792,982, filed Mar. 11, 2013; and U.S. Pat. Nos. 5,989,252 and 6,511,498, the entireties of which are incorporated by reference herein as if fully set forth herein and all of which are assigned to the same entity as the present invention.

FIG. 1C illustrates an undercut hole 68 formed by undercutting drill 10. The undercut hole 68 is a blind hole that may extend into bone 70. The inside diameter of the hole 68 is undercut by the cutting arm 46 to form a recessed space and overhanging surface 72, which overhangs the recessed space. The recessed space and overhanging surface 72 have certain advantages. One such advantage is that a filamentary anchor may be expanded into the recessed space, wherein the overhanging bone surface 72 may oppose movement of the filamentary anchor out of the undercut hole 68. Of course, if an alternative variation of drill tip 44 and cutting arm 46 would result is differing shapes of bone holes 68.

Another aspect of the present disclosure is a method for forming an undercut hole in bone which is partially illustrated by 1B. An undercut hole may be advantageous in many surgical applications, which may include soft tissue replacement or repair procedures, such as rotor cuff reparation, where tensioning and anchoring soft tissue to bone is desired.

In one embodiment, an incision may be made through soft tissue 74 to gain access to the portion of bone 70 selected for drilling. As illustrated in 1B, the second portion 24 of the drill guide 12 may be inserted through the soft tissue 74 and moved into a position in which the end of the second portion 24 abuts or lies flush against the bone 70. The distal end of drill guide 12 can have a serrated texture, or other shape, for improved grip on the bone surface. Alternatively, the second portion 24 of the drill guide 12 can be moved into such position through an arthroscopic cannula, which is inserted through the soft tissue 74. Generally, the first portion 22 of the drill guide 12 resides external to the patient's body and is fixed from rotational movement (from a mechanical connection to the torque applying device such as an electric drill) by, for example, a clip, screw threads, or a spring detent. In some embodiments, the drill guide 12 may be restricted from movement by a hand grip.

With or without the plunger 16 connected to the torque applying device, the drill tip 44 of the drill body 14 is positioned into and through the drill guide until the distal tip 44 contacts the surface of bone 70. Alternatively, the drill body can already be positioned within the drill guide, at least partially, prior to positioning the drill guide against the bone. The operator may then engage the torque applying device, if not already engaged, and the operator delivers a torque to the plunger 16. As the plunger 16 begins to rotate, the pin 38 presses against the drill body 14 via the longitudinal slots 36, thereby transferring the torque applied to the plunger 16 to the drill body 14. The drill body 14 and plunger 16 rotate in unison.

Thereafter, the operator may advance the drill body 14 through the drill guide 12, such that the drill tip 44 penetrates the bone, by applying a push force to the plunger 16. The push force is delivered to the spring 18 from the retainer 20. The spring 18 then transfers the push force to the retaining surface 42 and consequently the drill body 14. The push force is sufficient to advance the drill body 14 through the drill guide 12 and into the bone 70 but not to compress the spring 18.

Once the drill tip 44 has penetrated the bone 70 to a predetermined depth, the shoulder 34 of the drill body 14 abuts the shelf 27 of the drill guide 12 effectively preventing further distal movement of the drill body 14. While distal movement may be prevented by this shoulder-shelf engagement, the interface between the shoulder 34 and shelf 27 allows the drill body 14 to continue to rotate within the drill guide 12 and bone 70.

The operator may then continue to apply a push force to the plunger 16 (this force may be greater than the push force utilized to advance the drill body 14 into the bone 70) which may now be sufficiently large to compress the spring 18. As the spring 18 is compressed, the plunger 16 advances through the aperture 30 of the drill body 14 and the pin 38 slides along the longitudinal slots 36. As the plunger 16 is advanced, the blunt end 66 of the plunger 16 slides along the rounded convexity 62 of the depression 60. The bluntness of the plunger 16 and the roundness of the depression 60 are such that the cutting arm 46 gradually extends from the drill body 14 providing the operator feedback and control over the rate at which the cutting arm 46 extends.

The cutting arm 46 rotates about the pivot axis 58 and twists about the cutting arm's longitudinal axis so that the cutting surfaces and cutting tip 56 extend from the drill body before the remainder of the cutting arm 46. As the bone 70 pushes back against the cutting arm 46 and the plunger 16 presses against the rounded convexity 62, the depression 60 flexes outwardly toward the bone 70, which reduces the radius of the convexity 62 and concavity 64. This reduction in radius translates into an extension in length of the cutting arm 46 and also helps dampen the rate at which the cutting arm 46 is extended. The twisting action also reduces the angle of the second cut 51 with respect to the transverse axis which facilitates the removal of bone by the cutting surface of the second cut 51 to form an overhanging surface 72.

The operator may advance the plunger 16 until the slot and pin arrangement 36, 38 prevents further distal advancement. The slot may have a length suitable to allow the plunger 16 to advance a predetermined amount which correlates to a predetermined amount of undercut within bone hole 68. Further, the operator may make manual adjustments based on environmental factors such as the strength of the bone, density of bone, size of bone anchor to be implanted, and the like, and as such can manually adjust the amount of applied push force. Once the desired undercut has been achieved, the operator can remove the force from the plunger, which allows the spring 18 to elongate. As the spring 18 elongates, the spring 18 presses on the retainer 20, which moves the plunger in a proximal direction through the aperture 30 of the drill body 14. As the plunger 16 retracts beyond the depression 60, the cutting arm 46 swings back into the drill body 14 via the cutting arm's natural bias. Thereafter, the drill 10 may be retracted from the patient.

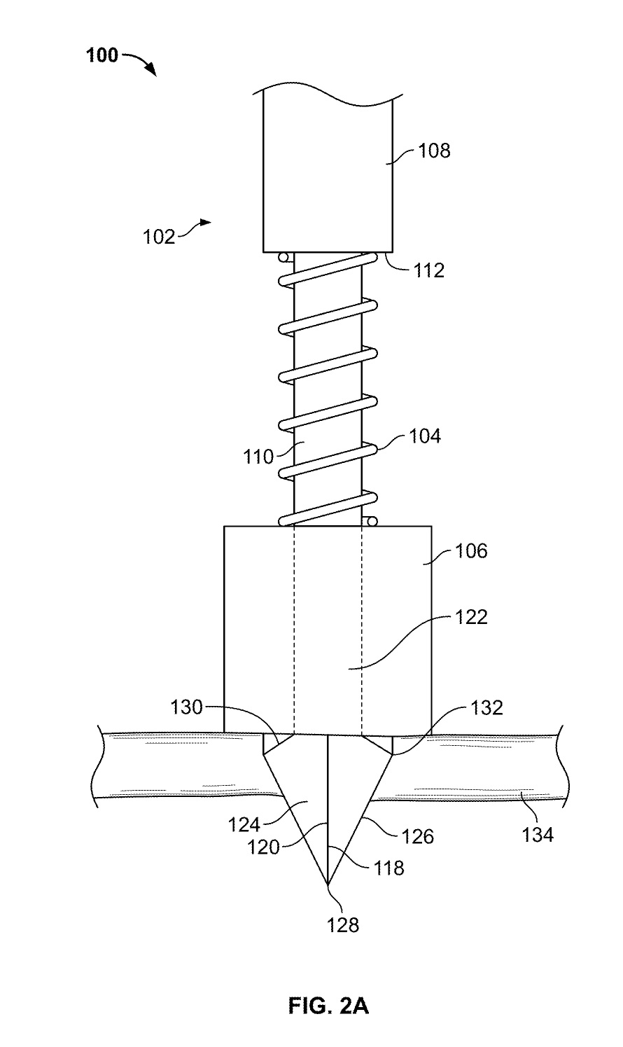

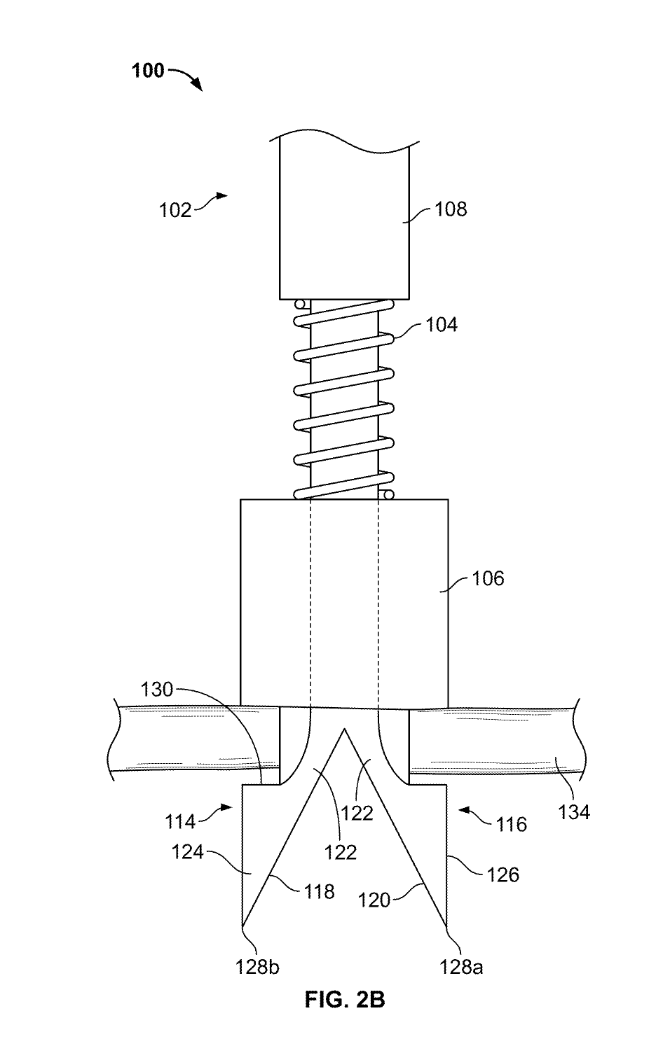

FIGS. 2A and 2B depict a second embodiment of an undercutting drill 100 that generally includes a drill shaft 102, a spring 104, or other biasing mechanism, and a bushing 106. The drill shaft 102 includes a proximal shaft 108 and a distal shaft 110. Both the proximal shaft 108 and distal shaft 110 are substantially cylindrical and are connected in a concentric arrangement. The proximal shaft 108 has an outer diameter larger than an outer diameter of the distal shaft 110. This difference in diameters forms an annular shoulder 112. The proximal shaft 108 is adapted to connect to a torque applying device, such as an electric drill, and is generally longer than the distal shaft 110. However, in some embodiments, the distal shaft 110 may be longer than or equal in length to the proximal shaft 108. The length of the distal shaft 110 is generally determined by the drill depth and the breadth of the undercut.

The distal shaft 110 has a distal end that is split symmetrically along a portion of its length to form a pair of cutter arms 114, 116 and respective inwardly-directed faces 118, 120. The cutter arms 114, 116 have an expanded configuration and a contracted configuration. In the contracted configuration, as depicted in FIG. 2A, the indwardly-directed faces 118, 120 can abut each other along their respective lengths. In the expanded configuration, as depicted in FIG. 2B, the cutter arms 114, 116 are splayed outwardly in a radial direction away from the longitudinal axis of the distal shaft 110 such that the inwardly-directed faces 118, 120 are separated and angled away from each other. In one example, when the cutter arms 114, 116 are in a state of static equilibrium, the cutter arms 114, 116 are in the expanded configuration. In other words, the cutter arms 114, 116 can be biased radially outwardly. The cutter arms 114, 116 may be moved into and held in the contracted configuration by a clamping force applied to each cutter arm 114, 116. The length of each inwardly-directed face 118, 120 is generally determined by the desired breadth of the undercut.

Each cutter arm 114, 116 includes a support member 122 and a cutting member 124. Each support member 122 is cantilevered to the distal shaft 110 and is semicylindrical such that each support member 122 has a radius. These radii are dimensioned such that when the cutter arms 114, 116 are in the contracted configuration, the support members 122 form a diameter that is substantially the same as the diameter of the remainder of the distal shaft 110.

The cutting members 124 are coupled to and supported by the support members 122. As best shown by FIG. 2A, each cutting member 124 is generally triangular and is defined by an outer cutting surface 126 and the inwardly-directed face 118 or 120 which extend distally towards a drill tip 128a, 128b. With the two cutting members in the contracted configuration, the cutting members 124 can form a generally kite-shaped quadrilateral that has a thickness that tapers distally and/or radially. This kite-shaped quadrilateral is defined by four sides including the two outer cutting surfaces 126 and shorter length sides 130. Further, each outer cutting surface and shorter length side can meet to form cutting tips 132 and, at a distal-most end, the two outer cutting surfaces can extend to form a drilling tip 128.

When in the contracted position, the distal drilling tip 128 can penetrate the bone and operate as a leading end for the drill, while the outer cutting surfaces 126 and cutting tips 132 can cut through bone to form the diameter of a blind hole prior to the undercut being formed. Once in the expanded position (best shown in FIG. 2B), the outer cutting surfaces 126 may be substantially parallel to each other, and the longitudinal axis of the drill shaft, and are the most distant points of the cutter arms 114, 116 in the radial direction such that they can cut through bone to help form the undercut.

The shorter length sides 130 may also include a cutting surface formed along their lengths such that they can cut through bone when the cutter arms 114, 116 are in the expanded position to form an overhanging surface that overhangs the undercut hole.

The cutting arms 114, 116 and distal shaft 110 are constructed from a biocompatible material that is flexible yet resilient such that arms 114, 116 can splay outwardly under their own natural bias and produce enough outward force to cut through bone in a radial direction while being able to retract to the position of FIG. 2A once preparation of the bone hole is complete. In one example, the material may be a memory metal alloy, such as Nitinol. Other examples include titanium, titanium alloys, stainless steel, cobalt-chromium, tantalum, and niobium. In one embodiment in which the distal shaft and cutting arms 114, 116 are constructed from a memory metal alloy, the cutting arms can be engineered to have an outwardly expanded shape memory such that when heat is generated during drilling, the shape memory is activated and the cutting arms become stiffer and/or bias toward outward expansion.

The bushing 106 is substantially cylindrical and includes a cylindrical passageway (not shown) extending therethrough such that the cylindrical passageway is concentric with the bushing's outer diameter. Examples of such bushings may be simple, cylindrically shaped objects, or may include an integrated spring portion, such as the bushings disclosed in U.S. Provisional Application No. 61/679,336, filed Aug. 3, 2012, and U.S. application Ser. Nos. 13/588,586 and 13/588,592, both of which filed Aug. 17, 2012, all of which are hereby incorporated by reference herein as if fully set forth herein. Of course, if the bushing itself includes an integrated spring portion, spring 104 may not be required. In some embodiments drill 100 may include multiple springs and bushings arranged in series.

The bushing's length may be determined by the drill depth where the spring 104, annular shoulder 112, and bushing 106 act as a depth stop. In some embodiments, the depth may be determined by a depth gauge etched into the proximal shaft 108 or by some other indicating means. In such embodiments, the length of the bushing 106 may not be determined by drill depth. The diameter of the cylindrical passageway is such that the bushing 106 can slide over the support arms 122 and clamp the cutter arms 114, 116 together so that the inwardly-directed faces 118, 120 abut each other in close proximity to minimize any gap that may be formed at the drill tip 128. The outer diameter of the bushing 106 may be dimensioned to fit within a drill guide and/or arthroscopic cannula.

The spring 104 may be a helical spring capable of fitting over the distal shaft 110 between the bushing 106 and annular shoulder 112. The spring 104 has a stiffness such that when the spring 104 is in a compressed state, the spring 104 can apply a force to the bushing 106 sufficient to slide the bushing 106 over the support arms 122 to move the cutter arms 114, 116 into and hold the cutter arms 114, 116 in the contracted configuration.

When the undercutting drill 100 is fully assembled, the spring 104 is disposed over the distal shaft 110 and is connected to and/or abuts the annular shoulder 112 at one end and is connected to and/or abuts the bushing 106 at the other end, which is also disposed over the distal shaft 110.

In the contracted configuration, as illustrated by FIG. 2A, the support arms 122 reside within the substantially cylindrical passageway of the bushing 106 and a portion of the cutting members 124 may also reside within the bushing 106. The bushing 106 clamps the cutter arms 114, 116 together such that the inwardly-directed faces 118, 120 abut each other along their length. The spring 104 is elongated relative to its compressed position when the cutter arms 114, 116 are in the expanded configuration. The spring 104 helps facilitate the cutter arms 114, 116 being held in the fully contracted position.

In a fully expanded configuration, as illustrated by FIG. 2B, the distal end of the bushing 106 is generally proximal of the support arms 122 and the spring 104 is compressed between the annular shoulder 112 and the bushing 106. The cutter arms 114, 116 are splayed outwardly under their own bias such that the inwardly-directed faces 118, 120 diverge and the longer length sides 126 are substantially parallel.

As previously mentioned, the drill depth may be determined by a visual indicator signaling to the operator when the proper depth has been reached. Alternatively, the drill depth may be determined by a mechanical stop wherein the drill shaft 102 is physically prohibited from further distal movement by the complete compression of the spring 104 and abutment of the annular shoulder 112 with the bushing 106 via the completely compressed spring 104.

In an alternative embodiment of undercutting drill 100, the distal shaft 110 may have more than two cutter arms 114, 116. For example, the undercutting drill may have three cutter arms, wherein the cutting members of the three cutter arms would be arranged in a pyramidal shape and the support members would each be a one-third segment of a cylinder that when combined would have the same diameter as the remainder of the distal shaft.

In another embodiment the undercutting drill 100 may not include a bushing 106. In such an embodiment, the cutter arms 114, 116 may be constructed from a memory metal alloy that would bias the cutter arms 114, 116 into the collapsed configuration at room and body temperatures, but would bias the cutter arms 114, 116 toward expansion into the expanded configuration at increasing temperatures, such as temperatures produced by friction between metal and bone.

In one aspect of the present disclosure, a method for forming an undercut hole in bone 134 with undercutting drill 100 is illustrated by FIGS. 2A and 2B. Referring to FIG. 2A, the cutter arms 114, 116 are in the contracted configuration and an incision is made in soft tissue to gain access to the portion of bone 134 selected for drilling. The undercutting drill 100 is inserted through the soft tissue or, alternatively, through an arthroscopic cannula inserted through soft tissue. In one embodiment, drill 100 may be placed through a drill guide prior to inserting drill 100 through soft tissue or an arthroscopic cannula.

The proximal shaft 108 is connected to a torque applying device, which applies a torque to rotate the undercutting drill 100. As the undercutting drill 100 is rotated, the operator places the drill tip 128 against the bone 134 and applies a push force to advance the cutting members 124 through the bone 134. The kite-shape of the cutting members 124 in the collapsed configuration allows the bone to be gradually resected as the cutting members 124 advance through the bone 134. Once the pair of cutter tips 132 penetrate and resect the bone 134 and before the undercut is formed, the widest diameter of the blind hole is achieved.

Referring to FIG. 2B, once the cutting members 124 have penetrated through the bone 134 to a predetermined depth, the bushing 106 contacts the bone surface effectively preventing any further distal movement of the bushing 106. Thereafter, the operator may apply a pushing force sufficient to compress the spring 104 and continue the advance of the cutting members 124 through the bone 134. As the spring 104 is compressed, the distal shaft 110 advances through the substantially cylindrical passageway of the bushing 106. The cutter arms 114, 116 gradually splay outwardly under their own bias as the support members 122 are unsheathed from the bushing 106. As the cutter arms 114, 116 splay outwardly, the cutting members 124 rotate away from each other such that the longer surfaces 126 advance toward a parallel position with respect to each other and the shorter surfaces 130 advance toward a position in which the shorter surfaces 130 reside along a transverse plane that is orthogonal to the longitudinal axis of the drill shaft 102. The short surfaces 130 form an overhang surface and the longer surfaces 126 form an undercut adjacent the overhang surface.

Typically, the undercutting is complete once the spring 104 is fully compressed and the bushing completely blocks further distal advancement of the drill.

Alternatively, the operator may manually decide when a sufficient amount of undercut is achieved. In either event, once the undercut is completed, the operator may retract the cutting device 100 from the undercut hole by reducing and/or reversing the push force. The reduction and/or reversing of the push force allows the spring 104 to advance the bushing 106 over the support arms 122, which gradually contracts the cutter arms 114, 116 into the contracted configuration. As the bushing 106 advances, the cutter arms 114, 116 swing back into the contracted configuration and are advanced through the undercut hole and removed from the patient.

Alternatively, using the embodiment without a bushing, and instead using cutting arms formed of memory metal, the slowing or complete cessation of the drilling operation decreases the temperature of the cutting arms such that they gradually return to the contracted position and the drill can be withdrawn from the patient.

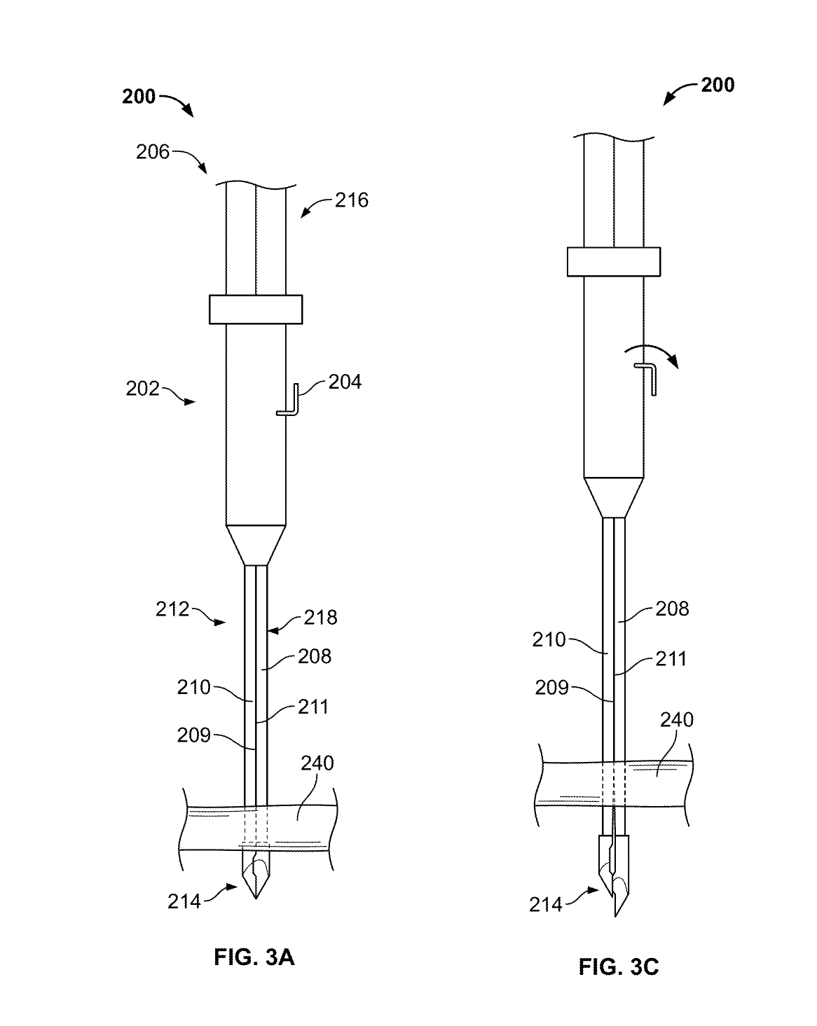

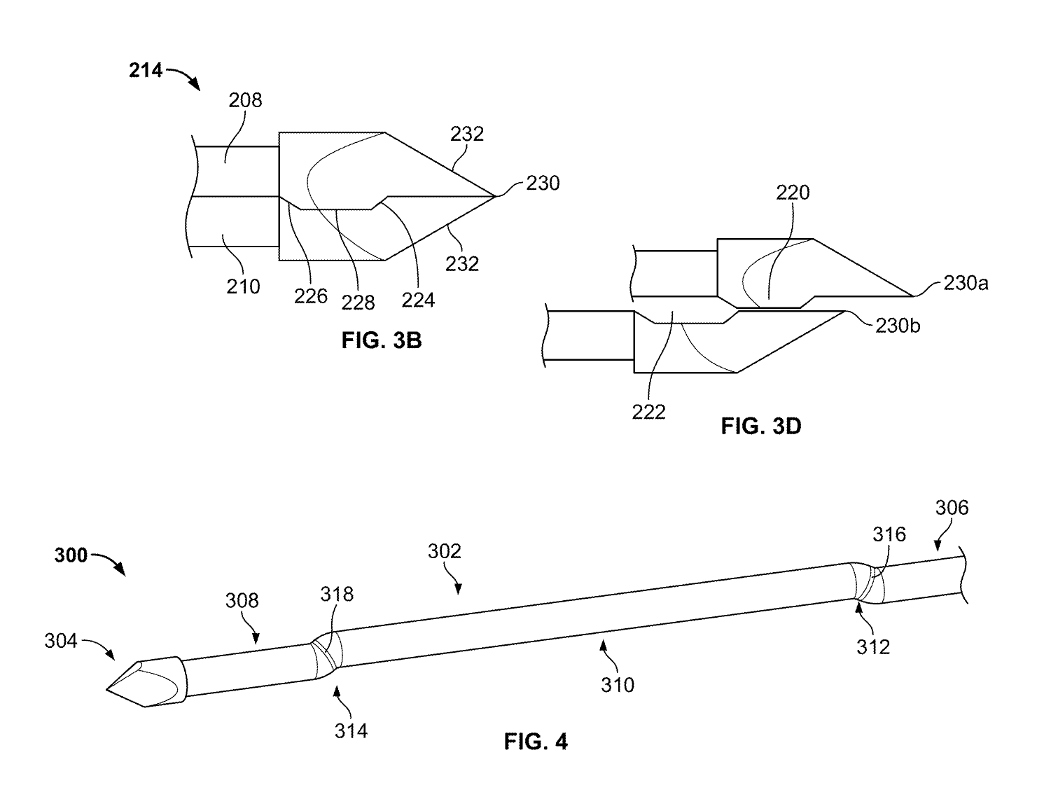

FIGS. 3A-3D illustrate yet another undercutting drill 200. Undercutting drill 200 generally includes a bushing 202, an actuation mechanism 204 and a split drill bit 206. The split drill bit 206 is a cylindrical drill bit that is split into a first segment 208 and a second segment 210 by a plane that bisects the drill bit's longitudinal axis. When the first and second segments 208, 210 are brought together into a working relationship, the first and second segments 208, 210 form a split shaft 212 and a split cutting tip 214. In the working relationship, the first and second segments 208, 210 may be in either a first condition or a second condition. In the first condition, the first and second segments 208, 210 are generally configured to drill a blind hole in bone 240. In the second condition, the first and second segments 208, 210 are generally configured to drill an undercut hole in bone 240.

In the first condition, as depicted by FIGS. 3A and 3B, the first and second segments 208, 210 interface at interior surfaces 209, 211 formed by the bisection of the drill bit 206 such that the split shaft 212 and split cutting tip 214 are cylindrical along their respective lengths. The split shaft 212 includes a proximal portion 216 and a distal portion 218. The proximal portion has a diameter sufficient to be chucked to a drill, such as a surgical bone drill or the like, and may be larger than the diameter of the distal portion 218. As the split shaft 212 is split through its longitudinal axis, the corresponding shafts of the first and second segment 208, 210 are semicylindrical.

The split cutting tip 214 includes a penetrating point 230 and cutting surfaces 232 and is similarly split through its longitudinal axis. As such, the penetrating point 230 is generally formed by the convergence of penetrating points 230a and 230b disposed at the distal end of the first and second segments, respectively, when in the first condition. In this condition, the outer diameter of the cutting tip 214 is preferably larger than the diameter of the distal portion 218 of the split shaft in order to provide clearance space in bone 240 when the drill bit 206 is moved from the first condition to the second condition.

Within the region of the cutting tip 214, the inner surface 209 of the first segment 208 includes a convexity 220, and the inner surface 211 of the second segment 210 includes a concavity 222 which is configured to substantially mate with the convexity 220 while in the first condition. The convexity 220 includes a converging surface 224, a diverging surface 226, and an offset surface 228 intermediate of the converging and diverging surfaces. These surfaces are generally planar, but can also be blended into a single curvilinear surface. Converging and diverging surfaces 224, 226 can have an interior angle of about 5 to 45 degrees with respect to the longitudinal axis. In some embodiments, the angles formed by the converging and diverging surfaces 224, 226 may be equal, while in other embodiments these angles may differ. For example, the converging surface 224 may have a relatively shallow angle (such as about 5 to 20 degrees), and the diverging surface 226 may have a relatively steep angle (such as about 25-45 degrees).

The bushing 202 may include an aperture extending therethrough to receive and retain drill bit 206. The bushing 202 may also include an actuation mechanism 204 that is connected to the first segment 208 and/or second segment 210. The actuation mechanism 204 may be configured to convert rotational motion to linear motion, such as by a geared mechanism, so that the rotation of a lever translates the first segment 208 axially in a distal direction and into the second condition. Other mechanisms may utilize linear movement, such as a thumb-slide.

FIGS. 3C and 3D depict the drill bit 206 in the second condition. In the second condition the penetrating tip 230a of the first segment 208 is located more distal than the penetrating tip 230b of the second segment 210. Also, the offset surface 228 abuts the inner surface 211 of the second segment 210 at a location distal of the concavity 222, which causes the first and second segments 208, 210 to bend slightly away from each other along the length of the split shaft 212.

In a method of using undercutting drill 200, the proximal portion 216 is chucked to a drill with the split drill bit 206 being in the first condition. A bore hole location is selected and a blind hole is formed by drilling into bone 240 while the drill bit 206 remains in the first condition. Once the desired depth is achieved, the actuation mechanism is engaged, which moves the first segment 208 (or in some embodiments second segment 210) axially. As the first segment 208 translates, the converging surface 224 pushes the first and second segments 208, 210 radially outwardly. The smaller diameter of the proximal portion 218 of the split shaft 212 provides clearance within the bore hole to allow the first and second segments 208, 210 to separate. As the segments separate, the cutting surfaces 232 form an undercut bore that has a diameter larger than the diameter of the blind hole initially created by the drill bit 206 in the first condition. The diameter of the undercut in relation to the blind hole may be determined by the distance the offset surface 224 is offset from the longitudinal axis of undercut drill 200. Once the undercut hole is formed, the actuation mechanism 204 may be actuated so that the convexity slides back into the concavity 222 placing the drill bit 206 back into the first condition for removal from bone 240.

Where the converging surface 224 has a relatively shallow angle, this angle may allow for a more gradual, radial expansion of the first and second segments 208, 2100 as first segment 208 is moved distally, which may make axial translation easier and help reduce dynamic loads. Where the diverging surface 226 has a relatively steep angle, the diverging surface 226 may help resist overshoot when first segment 208 is moved proximally back into the first condition.

FIG. 4 depicts another undercutting drill embodiment 300. Undercutting drill 300 generally includes a shaft 302 and a cutting tip 304. The cutting tip 304 preferably has a larger diameter than the shaft 302.

The shaft 302 includes a proximal shaft 306, a distal shaft 308, and an offset shaft 310. The distal shaft 308 extends from the cutting tip 304 and is generally coaxial with the proximal shaft 306 and cutting tip 304. The offset shaft 310 has an offset axis that is offset from and parallel to the longitudinal axis extending through the cutting tip 304, distal shaft 308, and proximal shaft 306.

The offset shaft 310 is offset from this longitudinal axis by a diverging bend 312 and a converging bend 314. The diverging bend 312 connects the proximal shaft 306 with the offset shaft 310 and includes a straight portion 316 that is angled relative to the longitudinal axis of the proximal shaft 306 by about 1 to 45 degrees. Straight portion 316 preferably has the same diameter as the proximal shaft 306 and is relatively short (between about 0 to 3 mm).

The converging bend 314 connects the distal shaft 308 with the offset shaft 310 and also may include a straight portion 318 that is angled relative to the longitudinal axis of the distal shaft 308 by about 1 to 45 degrees. The straight portion 318 of the converging bend 314 preferably has the same diameter as the distal shaft 308 and is relatively short (between about 0 to 3 mm). The angles and diameters of the diverging and converging bends 312, 314 may differ from one another but are preferably the same. Further, the diameter of the straight portion 314 (if present) of the converging bend 318 is preferably smaller than the diameter of the cutting tip 304.

In a method of using undercutting drill 300, the proximal shaft 306 is chucked to a drill, such as a surgical bone drill or the like. Thereafter, the cutting tip 304 is drilled into bone to form a blind bore hole the same diameter as the cutting tip 304. Once the desired depth is achieved, generally determined by the combined length of the cutting tip 304 and distal shaft 308, the converging bend 314 contacts the edge of the bore hole and bone surface adjacent the bore hole. At this point, the curvature of the converging bend 314 and the rotation of the drill begins to shift the center of rotation to the long axis of the offset shaft 310. In some embodiments, the center of rotation is shifted to the straight portion 318 of the converging bend. As the center of rotation is shifted, the cutting tip 304 and distal shaft 308 begin to wobble. As this occurs, the cutting tip forms an undercut hole within the blind hole, and the smaller diameter of the distal shaft 308 provides clearance space while also providing a limit to the expanse of the undercut hole so as to not over-penetrate. Once the undercut hole is reamed, undercutting drill 300 may simply be pulled out of the bone.

In an alternative embodiment, offset shaft 310 may include a tapered portion (not shown) at the distal end such that the diameter increases in a proximal direction until it is equivalent to the diameter of cutting tip 304. In this embodiment, offset shaft 310 can achieve a line-to-line fit with the bone hole once it is advanced such that the tapered portion is fully seated within the bone hole allowing for increased wobbling of cutting tip 304.

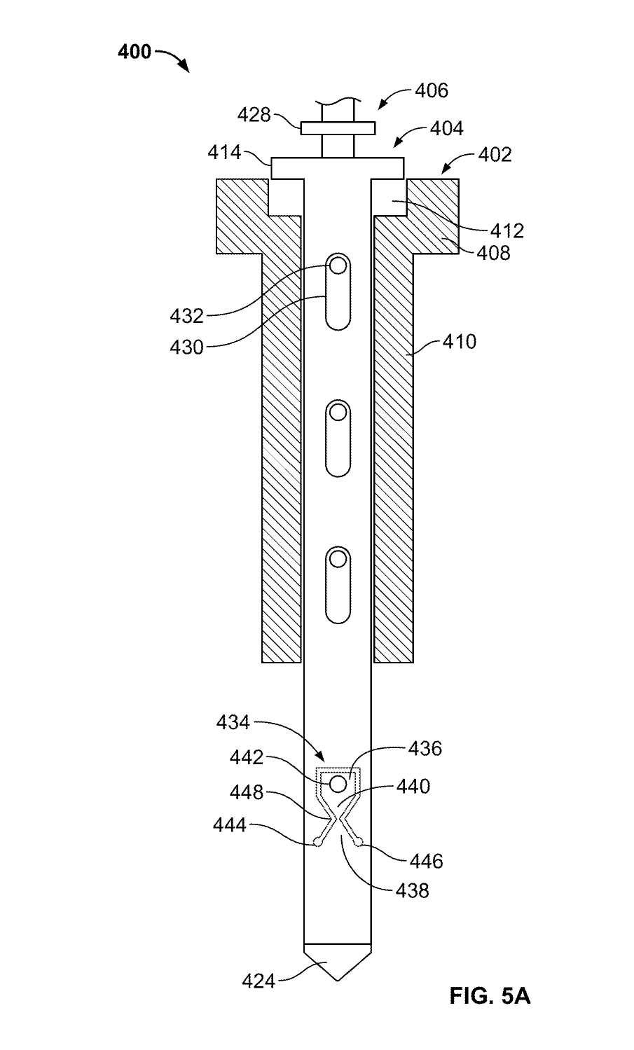

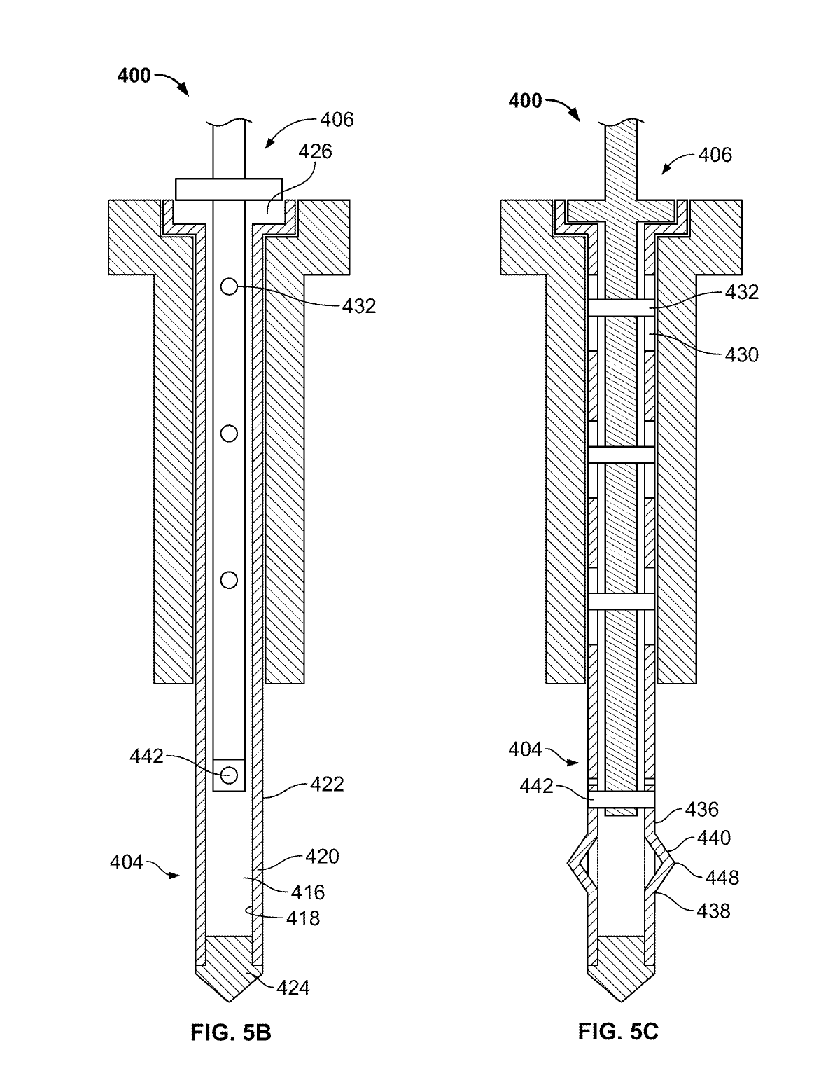

FIGS. 5A-5C depict yet another undercutting drill system embodiment 400. Undercutting drill 400 is similar to undercutting drill 10 but differs primarily with regard to the undercutting feature and spring. Undercutting drill system generally includes a drill guide 402, a tubular drill body 404, and a plunger 406.

The drill guide 402 is similar to drill guide 12 and includes an annular proximal portion 408 and an annular distal portion 410. The annular proximal portion 408 has a diameter larger than the annular distal portion 410 and includes an annular recess 412 configured to receive an annular flange 414 of the tubular drill body 404. The distal portion 410 may be configured for passage through an arthroscopic cannula and may be sufficiently long to allow the distal portion 410 to be placed against bone while simultaneously being controlled by the operator extracorporeally.

The tubular drill body 404 includes a channel 416 extending therein which is defined by an inner surface 418 of the drill body 404. As the body 404 is tubular, the body 404 has a sidewall 420 that extends between the outer surface 422 and inner surface 418. The body 404 also includes an annular flange 414 at the proximal end for rotation within the annular recess 412 of the bearing 402, and a drill tip 424 at the distal end for drilling a blind hole in bone. Annular flange 414 may also include a recess 426 for receipt of an annular flange 428 of the plunger 406. Further, the tubular body 404 includes a plurality of longitudinal slots 430 that extend through the entirety of the body 404 for receiving and guiding pins 432 that also pass through the plunger 406.

An undercutting feature 434 is formed by cutting, such as by laser cutting or the like, an hourglass-type pattern through the sidewall 420 of the drill body 404. The undercutting feature 434 generally includes a free end 436, a cantilevered end 438 (or fixed end), and a cutting region 440 disposed therebetween. The free end 438 is connected to the plunger 406, such as by pin 442 or other mechanical connection, and may have a square-like shape. In some embodiment, the free end 436 can take on other shapes, such as an arc shape or the like. The cantilevered end 438 is disposed opposite the free end 436 and may have a first circular hole 444 and a second circular hole 446 at the boundary of the cantilevered end 438 to help reduce stress concentrations during operation. The cutting region 440 is formed by straight cuts that taper inwardly from the free end 436 and from the cantilevered end 438 which converge to form a narrow neck region 448. In one embodiment, these cuts may be made at an oblique angle with respect to the outer surface of tubular body 404 to create an inclined cutting edge. The narrowed neck region 448 has a smaller cross sectional area than any other portion of the undercutting feature 434, which helps ensure that the undercutting feature bends 434 at the neck region 448 during operation.

In operation, with the drill body 404 extending through the drill guide 402 and the plunger 406 extending into the channel 416, the undercutting feature 434 moves from a first condition to a second condition by the distal translation of the plunger 406. In the first condition, the undercutting feature 434 conforms to the tubular construction of the drill body 404 and is generally flush with the inner and outer surfaces 418, 422. In the second condition, with the plunger 406 in a more distal position, the free end 436 is positioned closer to the cantilevered end 438 and the cutting feature 440 is bent at the neck region 448, at an axis between the first and second circular holes 444, 446, and at an axis between the free end 436 and cutting region 440 such that the narrow neck region 448 extends outwardly from the drill body 404 to form a cutting tip, as best seen in FIG. 5C.

It is noted that undercutting drill 400, unlike undercutting drill 10, does not include a spring. In embodiment 400, the spring is substituted for by the spring constant of undercutting feature 434 as may be determined by the geometry of the undercutting feature 434, the resistance provided by the bone in the bore hole, and the material utilized in the construction of the tubular body 404, which may include stainless steel, titanium, Nitinol, cobalt-chromium, or the like. As such, the particular dimensions of the sidewall 420 and undercutting feature 434 may be optimized in light of the material chosen so that the initial driving force for penetration of the drill 400 into bone does not actuate the undercutting feature 434, but rather, a second larger driving force does actuate the feature 434 when at the appropriate depth in bone is reached. While undercutting drill 400 has just been described as not including a spring, it should be understood that in some embodiments a spring may be included and may function just as spring 18 functions in undercutting drill 10.

The plunger 406 is substantially cylindrical and includes an annular flange 428 near the proximal end of the plunger 406 and a plurality of pinholes extending through the plunger in a transverse direction with respect to the plunger's longitudinal axis for receipt of pins 432. At least one pinhole is disposed near the distal end of the plunger for receipt of pin 442 and for connection to the free end 436 of the undercutting feature 434.

In a method of using undercutting drill 400, the plunger 406 is chucked to a drill, such as a surgical bone drill or the like. The distal portion 410 of the drill guide 402 is placed against the bone at the desired location, and the drill is activated causing the torque to be transferred from the plunger 406 to the tubular drill body 414 via pins 432 disposed within longitudinal slots 430. Initially the pins 432 may be located at the proximal end of each slot 430. As the drill body 404 rotates, a first push force is applied, which slides the plunger 406 and tubular drill body 404 concurrently through the drill guide 402 and into the bone to form a blind hole. Once the annular flange 414 of the tubular drill body 404 completely enters into the recess 412 of the drill guide 402, the predetermined drilling depth is reached. Thereafter, a second push force larger than the first push force is applied causing the plunger 406 to slide within the channel 416 and the pins 432 to slide distally within the slots 430. An undercut hole is indicated as being achieved when the pins 432 contact the distal ends of the longitudinal slots 430 and/or the annular flange 428 of the plunger 406 completely enters into the recess 426 of the body 404.

During the second push force, the undercutting feature 434 transitions from the first condition to the second condition by a buckling effect harnessed by the geometry of the undercutting feature 434. As the plunger 406 moves distally, the pin 442 slides the free end 436 distally toward the cantilevered end 438 and the cutting region 440 begins to move radially outwardly, thereby gradually cutting away bone to form the undercut region in the bore hole. Once the bore hole has been formed, the push force may be reversed, aided by the spring constant of the undercutting feature 434, such that the pin 442 slides the free end 436 proximally until the undercutting feature 434 is back in the first condition and free to be removed from the bore hole.

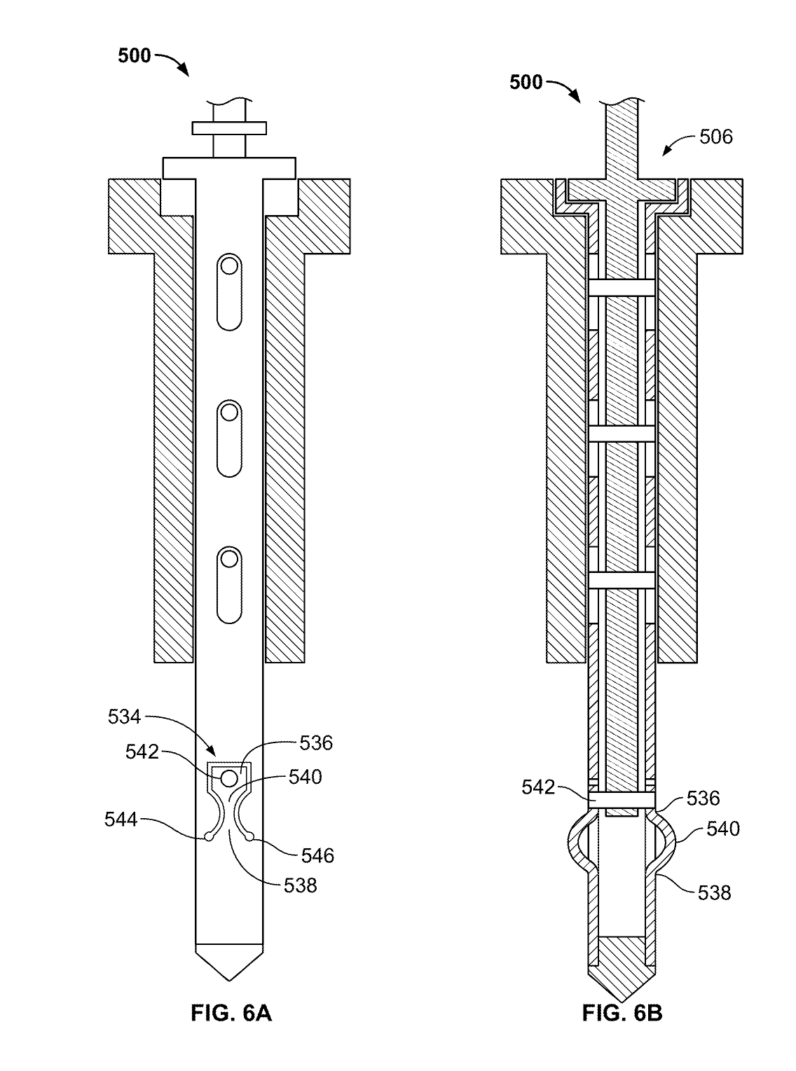

FIGS. 6A and 6B depict another alternative undercutting drill 500. Undercutting drill 500 is similar to undercutting drill 400 but differs with respect to the undercutting feature 534. Undercutting feature 534 similarly has an hourglass-type pattern and similarly includes a free end 536, a cantilevered end 538, and a cutting region 540. However, the cutting region 540 differs in that curved cuts form the cutting region 540, wherein each curved cut curves inwardly toward one another and each form an arch between the free end 536 and cantilevered end 544.

In operation, when the undercutting feature 534 is placed in the second condition, with the plunger 506 in a more distal position, the free end 536 is positioned closer to the cantilevered end 538 and the cutting feature 540 is bent at an axis between the first and second circular holes 544, 546, and at an axis between the free end 536. Further, while in the second condition, the cutting region is bowed outwardly from the drill body, as best seen by FIG. 6B. In order to take the undercutting feature 534 from a first condition to the second condition to form an undercut hole, the method of use is virtually the same as that of undercutting device 400 as previously described.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A bone cutting device for forming an undercut, comprising:
a rotatable body having an inner surface, and an outer surface, the inner surface defining an aperture, the inner and outer surfaces being disposed opposite each other and defining a sidewall, the sidewall having a recess extending from the outer surface into the aperture;
a cutting arm cantilevered to the sidewall and disposed within the recess, the cutting arm having a flexible depression extending into the aperture; and
an actuating member longitudinally slidable within the aperture,
wherein sliding the actuating member longitudinally interfaces with the flexible depression pushing the cutting arm radially outwardly.

2. The bone cutting device of claim 1, wherein the depression includes a concave portion having a first radius of curvature and a convex portion having a second radius of curvature, wherein applying a force to the concave portion reduces the size of the first and second radius and increases the length of the cutting arm.

3. The bone cutting device of claim 2, wherein the rotatable body includes a cutting tip disposed at a first end of the rotatable body and is configured to remove bone when a first force is applied to a second end of the rotatable body.

4. The bone cutting device of claim 3, further comprising a spring coupled to the actuating member at the second end of the rotatable body, the spring biasing the actuating member in a first direction and being compressible in a second direction by a second force.

5. The bone cutting device of claim 1, wherein the cutting arm is formed from at least three cuts that extend through the sidewall of the rotatable body.

6. The bone cutting device of claim 5, wherein:
the at least three cuts comprise a first cut, a second cut, and a third cut,
the first cut begins at a first point and extends along the rotatable body in a first direction,
the second cut begins at the end of the first cut and extends along the rotatable body in a second direction, and
the third cut begins at the end of the second cut and extends along the rotatable body in a third direction and ends at a second point.

7. The bone cutting device of claim 6, wherein:
the rotatable body has a longitudinal axis and a transverse axis orthogonal to the longitudinal axis,
the first direction is about 5 degrees or less with respect to the longitudinal axis of the rotatable body,
the second direction is about 5 to 10 degrees with respect to the transverse axis, and
the third direction is about 0 degrees with respect to the longitudinal axis.

8. The bone cutting device of claim 7, wherein the first cut and third cut are disposed on the same side of second cut.

9. The bone cutting device of claim 8, wherein the first cut and third cut are disposed distally of the second cut.

10. The bone cutting device of claim 9, wherein the first point is offset from the second point in a proximal-distal direction.

11. A bone cutting device for forming an undercut in bone, comprising:
an elongate shaft having a longitudinal axis;
a plurality of cutting arms cantilevered to the elongate shaft and having a first position and second position; and
a bushing slidable over the elongate shaft and at least a portion of the cutting arms,
wherein the bushing is adapted to slide between a distal position, over the cutting arms, which moves the cutting arms into the first position and a proximal position, away from the cutting arms, which moves the cutting arms into the second position.

12. The bone cutting device of claim 11, wherein the cutting arms extend away from the longitudinal axis in the second position and converge toward the longitudinal axis in the first position.

13. The bone cutting device of claim 12, wherein the cutting arms are biased towards the second position.

14. The bone cutting device of claim 12, wherein, at a first temperature, the cutting arms are biased towards the first position, and at a second temperature, the cutting arms are biased towards the second position.

15. The cutting device of claim 11, wherein the bushing is biased towards the distal position over the cutting arms.

16. The cutting device of claim 15, wherein the cutting arms, in the first position, form a distal cutting tip including a distal-most point and a cutting surface, along each cutting arm, having an enlarging taper expanding to a location on the cutting tip, proximal of the distal-most point, having a width larger than a width of the elongate shaft.

17. The cutting device of claim 16, wherein the bushing is prevented from sliding distally to a position passed the width of the cutting tip that is larger than the width of the elongate shaft.

18. The cutting device of claim 16, wherein, when the cutting arms are in the second position, the cutting surface is substantially parallel with the longitudinal axis of the elongate shaft.

19. A bone cutting device for forming an undercut, comprising:
a tubular drill body having an inner surface and outer surface defining a sidewall therebetween and a drill tip disposed at one end of the tubular drill body; and
a cutting arm formed from the sidewall by at least three cuts through the sidewall, the cutting arm having a cantilevered end, a free end, and a cutting surface disposed between the cantilevered and free ends.

20. The bone cutting device of claim 19, further comprising a plunger having a length and being disposed within the tubular drill body and coupled to the tubular drill body at a location along the length of the plunger.
